(19) United States
(12) Patent Application Publication
(10) Pub. No.: US 2003/0195330 A1
(43) Pub. Date: Oct. 16, 2003

(54) Functionalized $g(p)-conjugated polymers, based on 3,4-alkylenedioxythiophene

(76) Inventors: Lambertus Groenendaal, Sinaai (BE); Knud Reuter, Krefeld (DE); Peter Bauerle, Thalfingen (DE); Alexander Meyer, Krefeld (DE)

Correspondence Address: Bayer Corportion, Patent Department, 100 Bayer Road, Pittsburgh, PA, 15205-9741, US

(21) Appl. No.: 10/296,570

(22) PCT Filed: May 10, 2001
(86) PCT No.: PCT/EP01/05362

(30) Foreign Application Priority Data
May 23, 2000 (DE) 10025309.1

(51) Int. Cl. C07D 333/02; C08G 75/00

(52) U.S. Cl. 528/373; 549/29

(57) ABSTRACT

Polythiophenes of the formula II

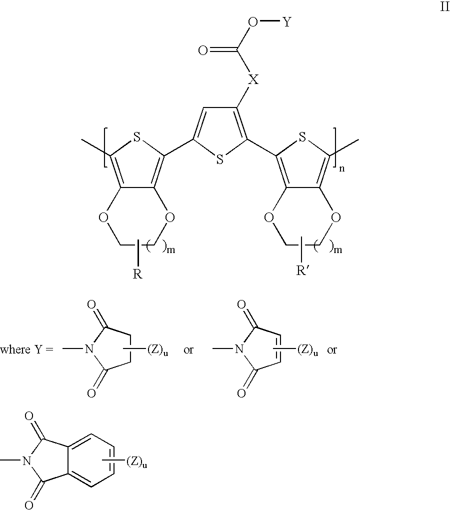

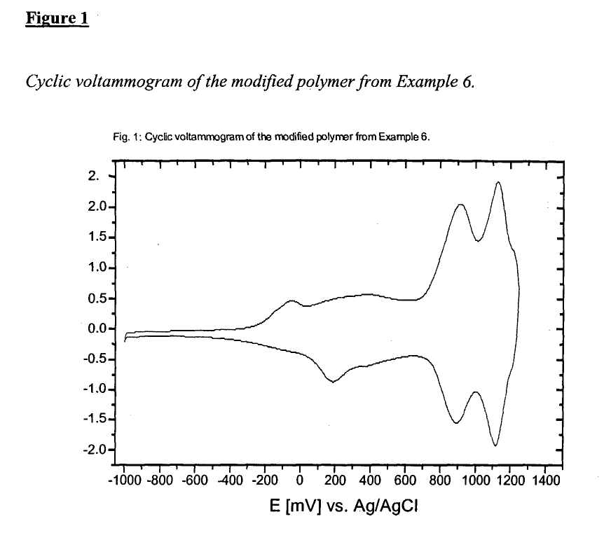

Z=alky, aryl or alkylaryl,

u=0 or 1,

m=0 to 5,

n=2 to 500,

X=(CH₂)_{p+q} or (CH₂)ₚ—O—(CH₂)_{q},

p, q are each, independently of one another, from 0 to 10 and

R, R′ are identical or different and are each, independently of one another, H, a linear or branched C₁-C₁₈ alkyl or alkoxy radical or a linear or branched C₁-C₁₈ alkylsulfonate or alkoxysulfonate radical, can be modified via the active ester function and have excellent electrical and optical properties.

The invention relates to new π-conjugated polymers based on 3,4-alkylenedioxythiophene and functionalized with active ester groups, their preparation from the monomers and their modification by means of active ester function.

π-Conjugated polymers as a class have been the subject of numerous publications in recent decades. They are also referred to as conductive polymers or as synthetic metals.

Owing to the considerable delocalization of the π electrons along the main chain, these polymers display interesting (nonlinear) optical properties and after oxidation or reduction they are good electric conductors. As a result, these compounds are expected to assume a leading and active role in various practical applications, e.g. in data storage, in optical signal processing, in the suppression of electromagnetic interference (EMI) and in solar energy conversion, and also in rechargeable batteries, light-emitting diodes, field effect transistors, circuit boards, sensors and antistatic materials.

Examples of known π-conjugated polymers are polypyrrols, polythiophenes, polyanilines, polyacetylenes, polyphenylenes and poly(p-phenylene-vinylenes).

Particular properties can be set by introduction of substituents on the monomer units. However, a problem with most functionalized π-conjugated polymers is that it is difficult to alter the substituents and thus the properties.

We have therefore prepared novel π-conjugated polymers based on 3,4-alkylenedioxythiophene and functionalized with active ester groups. By means of this active ester group, it is possible for numerous new substituents which control the properties and thus the range of uses of these polymers to be bound covalently to the polymers. The first preparation of such polymers based on 3,4-alkylenedioxythiophenes represents a technical step forward especially since this specific class of polythiophenes has excellent electrical and optical properties (EP-A-339 340).

π-Conjugated polymers functionalized with active ester groups are known. Bäuerle et al. (Adv. Mater. 1996, 8, 214-218) prepared polythiophenes functionalized with active ester groups.

π-Conjugated polymers based on 3,4-alkylenedioxythiophene and functionalized with active ester groups are not yet known. The conversion of these π-conjugated polymers functionalized with active ester groups into new functionalized π-conjugated polymers based on 3,4-alkylenedioxythiophene and the parent monomers are new.

The invention firstly provides terthiophenes of the formula I:

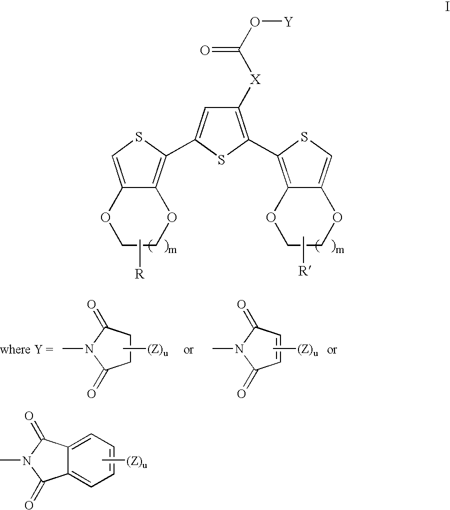

Z=alkyl, aryl or alkylaryl,

u=0 or 1,

m=0 to 5,

X=(CH₂)_{p+q} or (CH₂)ₚ—O—(CH₂)_{q},

p, q are each, independently of one another, from 0 to 10 and

R, R′ are identical or different and are each, independently of one another, H, a linear or branched C1-C18 alkyl or alkoxy radical or a linear or branched C1-C18 alkylsulfonate or alkoxysulfonate radical,

which are suitable for preparing functionalized π-conjugated polymers based on 3,4-alkylenedioxythiophene.

Preference is given to a monomer of the formula I in which

m=1,

X=(CH₂)₅ and

R, R′=H.

Alkyl is preferably C₁-C₁₈-alkyl and aryl is preferably C₆-C₁₀-aryl.

Scheme 1 shows the synthesis of N-{6-[2,5-bis(3,4-ethylenedioxythien-2-yl)thien-3-yl]hexanoyloxy}pyrrolidine-2,5-dione (5), starting from 6-(thien-3-yl)hexanoic acid (1). Compound 1 has been described by Bäuerle et al. in Adv. Mater. 1996, 8, 214-218. Examples 1 to 4 describe the synthesis of the monomer 5.

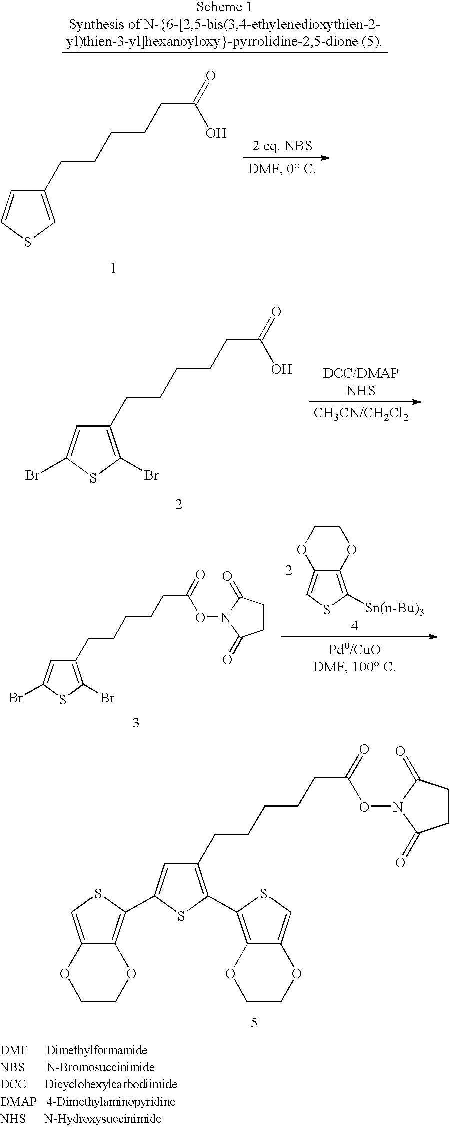

The invention also provides a process for the electrochemical polymerization of the monomers of the formula I. This electropolymerization can take place in various solvents (preferably in CH₂Cl₂ or acetonitrile) in the presence of various electrolytes (preferably tetrabutylammonium hexafluorophosphate or tetrabutylammonium perchlorate).

Instead of the electrochemical polymerization, the abovementioned monomers can also be polymerized by a chemical oxidative method, which is likewise subject matter of the invention. Suitable oxidants for the chemical polymerization of the abovementioned monomers are, for example, Fe(III) salts, in particular FeCl₃, H₂O₂, K₂Cr₂O₇, K₂S₂O₈, Na₂S₂O₈, KMnO₄, alkali metal perborates and alkali metal or ammonium persulfates. Further suitable oxidants are described, for example in Handbook of Conducting Polymers (Ed. Skotheim, T. A.), Marcel Dekker: New York, 1986, Vol. 1, 46-57.

These processes can, for example, be carried out in a manner analogous to the methods disclosed in EP-A-339 340.

The invention further provides polythiophenes of the formula II which can be prepared by electrochemical or chemical oxidative polymerization of the monomeric terthiophenes of the formula II:

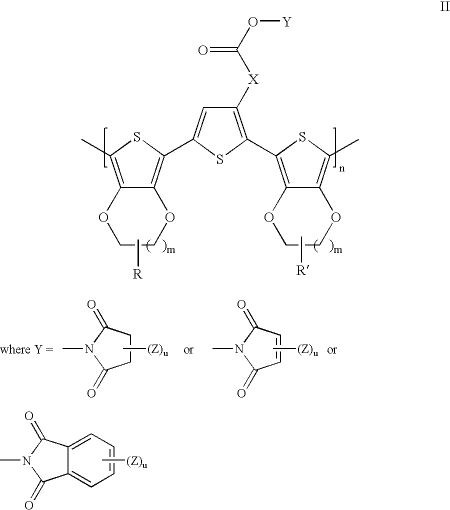

Z=alkyl, aryl or alkylaryl,

u=0 or 1,

m=0 to 5,

n=2 to 500,

X=(CH₂)_{p+q} or (CH₂)ₚ—O—(CH₂)_{q},

p, q are each, independently of one another, from 0 to 10 and

R, R′ are identical or different and are each, independently of one another, H, a linear or branched C1-C18 alkyl or alkoxy radical or a linear or branched C1-C18 alkylsulfonate or alkoxysulfonate radical.

Preference is given to a polymer of the formula II in which

m=1,

n=2 to 200,

X=(CH₂)₅ and

R,R′=H.

Particular preference is given to a polymer of the formula II in which

m=1,

n=2 to 200,

X=(CH₂)₅ and

R,R′=H.

The electrochemical polymerization of N-{6-[2,5-bis(3,4-ethylenedioxythien-2-yl)thien-3-yl]hexanoyloxy}pyrrolidine-2,5-dione (5) will be described as Example 5.

Finally, the invention provides for the modification of the polymers prepared electrochemically or by chemical oxidation or layers of the π-conjugated polymers of the formula II by chemical reaction, in particular the reaction of the active ester with amines to form amides of the formula III.

These modification reactions give polymers of the formula III, which are likewise subject matter of the invention:

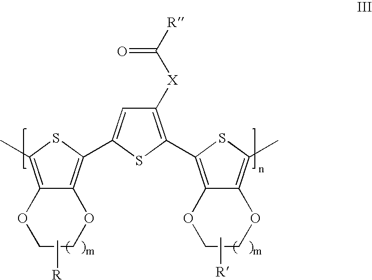

where

m=0 to 5,

n=2 to 500,

X=(CH₂)_{p+q} or (CH₂)ₚ—O—(CH₂)_{q},

p, q are each, independently of one another, from 0 to 10 and

R, R′ are identical or different and are each, independently of one another, H, a linear or branched C1-C18 alkyl or alkoxy radical or a linear or branched C1-C18 alkylsulfonate or alkoxysulfonate radical and

R″=an oligonucleotide radical, e.g.

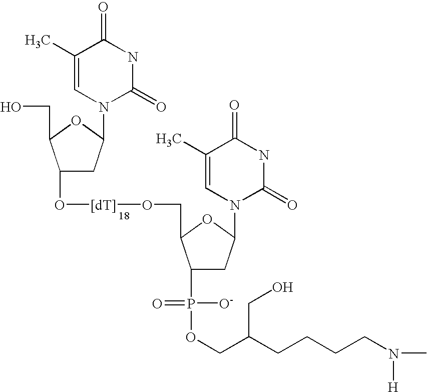

where dT=deoxythymidine monophosphate

or a radical selected from the group consisting of metal-free or metal-containing porphyrins, functionalized ferrocenes and functionalized calixarenes, e.g.

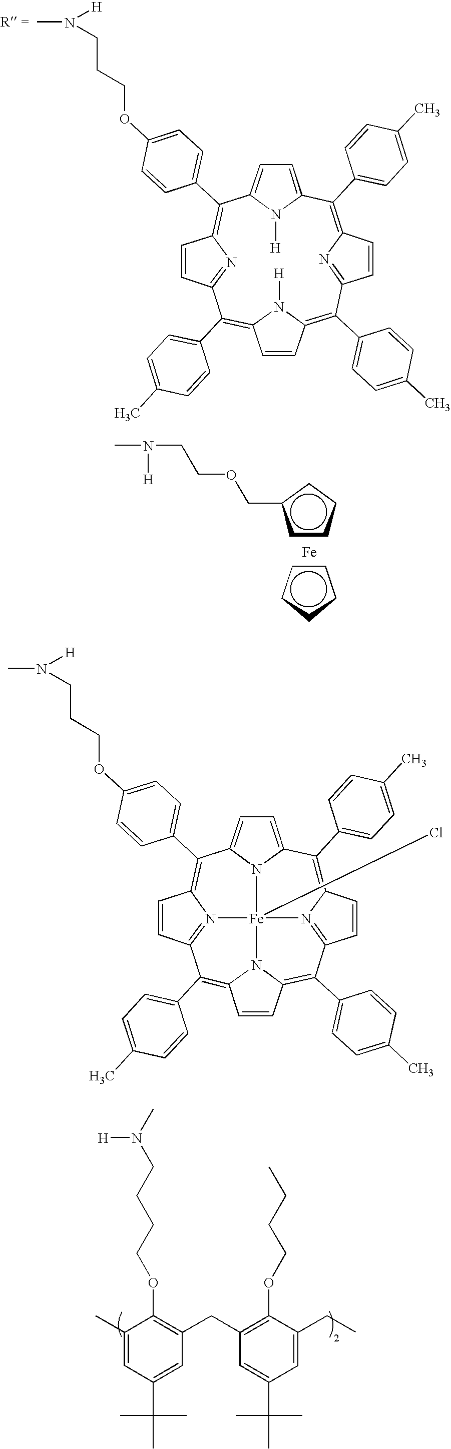

Particular preference is given to polymers of the formula III in which

m=1,

n=2 to 200, preferably from 2 to 20,

X=(CH₂)₅,

R,R′=H and

R″=an oligonucleotide radical, e.g.

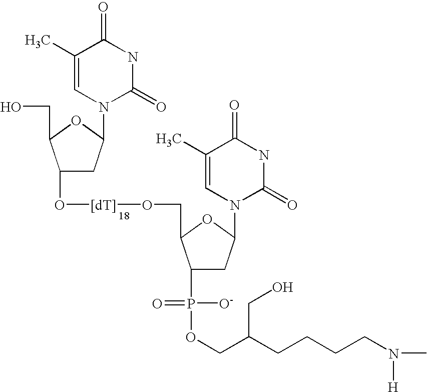

where dT=deoxythymidine monophosphate

or a radical selected from the group consisting of metal-free or metal-containing porphyrins, functionalized ferrocenes and functionalized calixarenes, e.g.

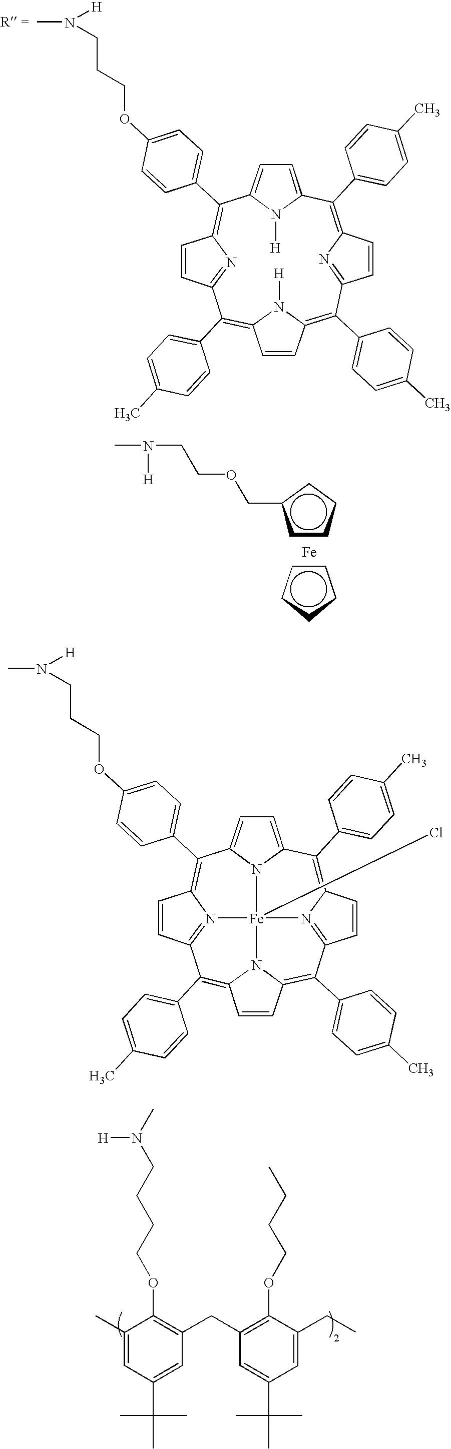

Modification methods for preparing the abovementioned polymers can be formulated on the basis of the work of Bäuerle et al. (Adv. Mater. 1996, 8, 214-218; Adv. Mater. 1996, 8, 219-222). The modification of poly(N-{6-[2,5-bis(3,4-ethylenedioxythien-2-yl)thien-3-yl]hexanoyloxy}pyrrolidine-2,5-dione (5) by means of an amino-substituted porphyrin derivative (for the preparation of this compound, see Meunier et al. Tetrahedron, 1989, 45, 2641-2648) will be described as Example 6.

All the abovementioned monomers and polymers can be readily characterized by techniques such as UV spectroscopy, fluorescence spectroscopy, infrared spectros-copy, NMR spectroscopy, mass spectrometry, cyclic voltammetry (see Example 6) and/or X-ray structural analysis.

Particular important applications for the π-conjugated polymers based on 3,4-alkylenedioxythiophene are, for example:

data storage,

optical signal processing,

suppression of electromagnetic interference (EMI),

solar energy conversion,

rechargeable batteries,

light-emitting diodes,

field effect transistors,

sensors,

antistatic materials and

through-contacts in printed circuits and multilayers.

### EXAMPLES

### Example 1

Synthesis of 2,5-dibromo-6-(thien-3-yl)hexanoic acid (2)

A solution of 2.92 g (16.4 mmol) of NBS in DMF (50 ml) is slowly added dropwise at 0° C. to a solution of 6-(thien-3-yl)hexanoic acid 1 (1.63 g, 8.20 mmol) in DMF (50 ml) with exclusion of light. The solution is stirred for 48 hours and then poured onto ice. The mixture is extracted a number of times with dichloromethane. The organic phases are combined, washed with water and dried over sodium sulfate. After removal of the solvent, the crude product is chromatographed on silica gel using n-hexane/ethyl acetate (1:1) as eluent (flash chromatography). This gives 2.57 g of 2 (88%) as an orange viscous oil; C₁₀H₁₂O₂SBr₂ Calc.: C, 33.73; H, 3.40. found: C, 33.56; H, 3.40. ¹H-NMR (200 MHz, CDCl₃): δ_{H}=6.77 (s, 1 H, 4′-H), 2.52 (t, ³J=7.9 Hz, 2 H, 6-H ), 2.37 (t, ³J=7.9 Hz, 2 H, 2-H), 1.75-1.51 (m, 4 H, 3-H, 5-H), 1.45-1.33 (m, 2 H, 4-H); ¹³C-NMR (126 MHz, CDCl₃): δ_{C} 179.3 (C-1), 142.5, 131.0, 110.5, 108.1 (C-2′-C-5′), 33.8, 29.2 (2 C), 28.4, 24.3 (C-2-C-6).

### Example 2

Synthesis of N-[6-(2,5-dibromothien-3-yl)hexanoyloxy]pyrrolidine-2,5-dione (3)

Under argon atmosphere, a solution of 1.38 g (6.70 mmol) of N,N′-di-cyclohexylcarbodiimide in 10 ml of acetonitrile is added dropwise at 0° C. to a solution of 2.39 g (6.70 mmol) of 6-(2,5-dibromothien-3-yl)hexanoic acid 2, 0.77 g (6.70 mmol) of N-hydroxysuccinimide and 16.4 mg (2 mol %) of 4-(N,N′-dimethyl)aminopyridine in 20 ml of acetonitrile/dichloromethane (1:1). The reaction mixture is stirred for 3 hours at 0° C. and then for a further 24 hours at room temperature. The insoluble residue is filtered off and the solvent is removed under reduced pressure. This gives a yellowish oil. The crude product is chromatographed on silica gel using ethyl acetate/n-hexane (1:1). This gives 2.31 g of 3 (76%) as a yellow viscous oil; C₁₄H₁₅Br₂NO₄S Calc.: C, 37.12; H, 3.34; N, 3.09; S, 7.07; found: C, 37.42; H, 3.40; N, 2.99; S, 7.26; ¹H-NMR (500 MHz, CDCl₃): δ_{H}=6.78 (s, 1 H, 4′-H) 2.84 (s, 4 H, 3″-H, 4″-H), 2.62 (t, ³J=7.3, 2 H, 2-H), 2.53 (t, ³J=7.6, 2 H, 6-H), 1.81-1.75 (m, 2 H, 3-H or 5-H), 1.62-1.58 (m, 2 H, 3-H or 5-H), 1.48-1.43 (m, 2 H, 4-H); ¹³C-NMR (126 MHz, CDC₃): δ_{C}=169.1 (2 C, C-3″, C-4″), 168.5 (C-1), 142.4, 130.9, 110.5, 108.1 (C-2′-C-5′), 30.8, 29.1, 29.0, 28.1, 25.6 (2 C), 24.3 (C-2-C-6, C-3″, C-4″).

### Example 3

Synthesis of 2-(tri-n-butylstannyl)-3,4-ethylenedioxythiophene (4)

1.99 g (14.0 mmol) of 3,4-ethylenedioxythiophene and 40 ml of THF are placed in a round-bottom flask under an argon atmosphere. After the solution has been cooled to 78° C., 8.75 ml (14.0 mmol) of n-butyllithium (1.6 M in n-hexane) are slowly added dropwise. The mixture is stirred for 15 minutes, warmed to room temperature and then stirred for another 60 minutes. The solution is then cooled to −70° C. and tri-n-butyltin chloride dissolved in 40 ml of THF is then slowly added dropwise by means of a syringe. The reaction mixture is warmed to room temperature and stirred for 14 hours. The solution is then carefully poured into cold water and extracted a number of times with diethyl ether. The combined organic phases are dried over magnesium sulfate and filtered through basic aluminum oxide. Unreacted starting material is removed under reduced pressure. This gives 4.18 g of 4 (69%) as colorless oil; C₁₈H₃₂O₂SSn Calc.: C, 50.14; H, 7.48; S, 7.44. found: C, 50.28; H, 7.48; S, 7.24. ¹H-NMR (500 MHz, CDCl₃): δ_{H}=6.56 (s, 1 H, 5-H), 4.14-4.11 (m, 4 H, 6-H, 7-H), 1.57-1.51 (m, 6 H, CH₂), 1.34-1.31 (m, 6 H, CH₂), 1.10-1.06 (m, 6 H, CH₂), 0.89 (t, ³J=7.0 Hz, 9 H, CH₃); ¹³C-NMR (126 MHz, CDCl₃): δ_{C}=147.7, 142.5, 108.9, 105.8 (C2-C5), 64.7, 64.6 (C6, C7), 29.0, 27.2, 13.7, 10.5 (n-butyl-C).

### Example 4

Synthesis of N-{6-[2,5-bis(3,4-ethylenedioxythien-2-yl)thien-3-yl]-hexanoyloxy}pyrrolidine-2,5-dione (5)

Under an argon atmosphere, 0.91 g (2.00 mmol) of N-[6-(2,5-dibromothien-3-yl)-hexanoyloxy]pyrrolidine-2,5-dione 3, 0.23 g (0.20 mmol) of Pd(PPh₃)₄ and 0.32 g (4.0 mmol) of CuO are dissolved in 10 ml of DMF and stirred at 100° C. After 5 minutes, a solution of 2.33 g (5.40 mmol) of 2-(tri-n-butylstannyl)-3,4-ethylenedioxythiophene 4 dissolved in 2 ml of DMF is added all at once by means of a syringe. The reaction is monitored by HPLC. After 8 hours, the halogen component has been consumed and the reaction mixture is slowly warmed to room temperature. The solution is poured onto ice and extracted a number of times with dichloromethane. The organic phases are combined, washed a number of times with water and dried over sodium sulfate. After removal of the solvent, the crude product is chromatographed on silica gel using ethyl acetate/n-hexane (2:1) as eluent. This gives 0.63 g (55%) of 5 as a yellow solid; C₂₆H₂₅NO₈S₃ Calc.: C, 54.25; H, 4.38; N, 2.43. found: C, 54.47; H, 4.64; N, 2.24. ¹H-NMR (500 MHz, CDCl₃): δ_{H}=7.04 (s, 1 H, 4′-H), 6.35 (s, 1 H, 5-H), 6.20 (s, 1 H, 5″-H), 4.33-4.21 (m, 8 H, Hᵢ, Hⱼ, Hₖ, Hₗ), 2.82 (s, 4 H, Hₕ, H_{h′}), 2.69 (t, ³J=7.9 Hz, 2 H, Hₐ), 2.59 (t, ³J=7.6 Hz, 2 H, Hₑ), 1.79-1.64 (m, 4 H, H_{b}, H_{d}), 1.50-1.45 (m, 2 H, H_{c}); ¹³C-NMR (126 MHz, CDCl₃): δ_{C}=169.1 (2 C, C_{g}, C_{g′}), 168.6 (C_{f}), 141.8, 141.5, 140.4, 138.2, 137.5, 134.0, 125.9 (thienyl-C), 124.9 (C-4′), 112.2, 110.2 (thienyl-C), 98.9, 96.8 (C-5, C-5″), 65.0, 64.8, 64.6, 64.5 (Cᵢ, Cⱼ, Cₖ, Cₗ), 30.9, 29.8, 29.0, 28.6, 25.5 (2 C), 24.3 (Cₐ-Cₑ, Cₕ, C_{h′}).

### Example 5

Electrochemical Polymerization of N-{6-[2,5-bis(3,4ethylenedioxythien-2-yl)-thien-3-yl]hexanoyloxy}pyrrolidine-2,5-dione (5)

5 ml of CH₂Cl₂/Bu₄NPF₆ (0.1 M) are purged with dry argon for 15 minutes. The monomer concentration of N-{6-[2,5-bis(3,4-ethylenedioxythien-2-yl)thien-3-yl]hexanoyloxy}pyrrolidine-2,5-dione 5 is 1·10⁻³ M. The electropolymerization is carried out by cyclic voltammetry in a prescribed potential range (to produce thin films of poly-(N-{6-[2,5-bis(3,4-ethylenedioxythien-2-yl)thien-3-yl]hexanoyloxy}pyrrolidine-2,5-dione): 20 cycles between −1.00 and +0.90 V vs. Ag/AgCl at a monomer concentration of 1·10⁻³ M). The working electrode treated in this way is rinsed with dry dichloromethane, dried in air and characterized electrochemically in monomer-free solution.

### Example 6

Modification of poly(N-{6-[2,5-bis(3,4-ethylenedioxythien-2-yl)thien-3-yl]hexanoyloxy}pyrrolidine-2,5-dione) with an Uncomplexed Porphyrin Derivative

The substitution of poly(N-{6-[2,5-bis(3,4-ethylenedioxythien-2-yl)thien-3-yl]hexanoyloxy}pyrrolidine-2,5-dione) is carried out by dipping the polymer layer into a THF solution of the amino-substituted porphyrin A. After 20-30 minutes at room temperature, the polymer layer is taken out and washed with absolute THF.

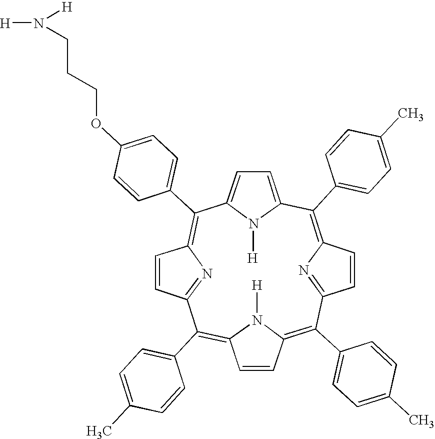

1. A monomeric terthiophene of the formula I 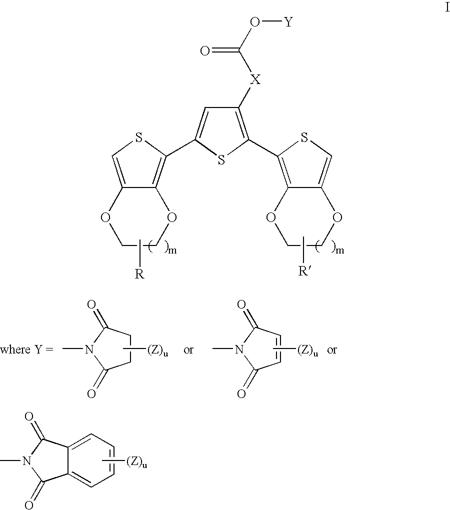
Z=alkyl, aryl or alkylaryl,
u=0 or 1,
m=0 to 5,
X=(CH₂)_{p+q} or (CH₂)ₚ—O—(CH₂)_{q},
p, q are each, independently of one another, from 0 to 10 and
R, R′ are identical or different and are each, independently of one another, H, a linear or branched C1-C18 alkyl or alkoxy radical or a linear or branched C1-C18 alkylsulfonate or alkoxysulfonate radical.

2. A monomeric terthiophene as claimed in claim 1, characterized in that
m=1,
X=(CH₂)₅ and
R,R′=H.

3. A process for the polymerization of monomeric terthiophenes as claimed in claim 1 or 2, characterized in that the terthiophenes are polymerized electrochemically.

4. A process for the polymerization of monomeric terthiophenes as claimed in claim 1 or 2, characterized in that the terthiophenes are polymerized by a chemical oxidative method.

5. A polythiophene of the formula II 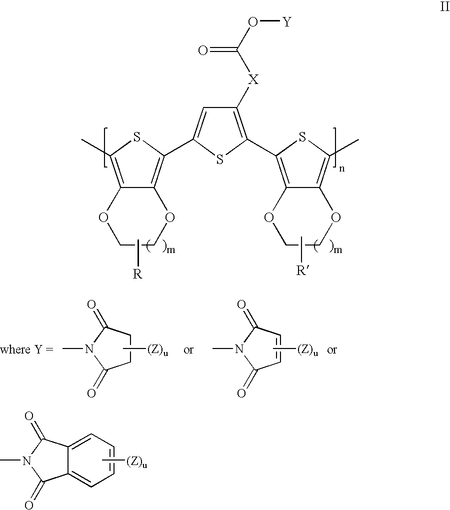
Z=alkyl, aryl or alkylaryl,
u=0 or 1,
m=0 to 5,
n=2 to 500,
X=(CH₂)_{p+q} or (CH₂)ₚ—O—(CH₂)_{q},
p, q are each, independently of one another, from 0 to 10 and
R, R′ are identical or different and are each, independently of one another, H, a linear or branched C1-C18 alkyl or alkoxy radical or a linear or branched C1-C18 alkylsulfonate or alkoxysulfonate radical.

6. A polythiophene as claimed in claim 5, characterized in that
m=1,
n=2 to 200,
X=(CH₂)₅ and
R, R′=H.

7. A process for the chemical modification of polythiophenes as claimed in claim 5 or 6, characterized in that they are reacted with at least one equivalent, based on the active ester groups in the polythiophenes, of a functional, in particular monoamino-functional, compound selected from the group consisting of oligonucleotides, porphyrins, ferrocenes and calixarenes.

8. A polythiophene of the formula III 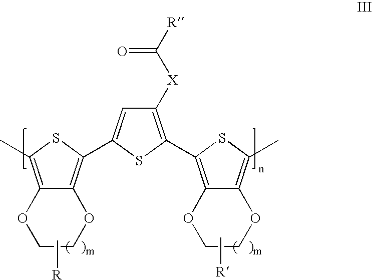
where
m=0 to 5,
n=2 to 500,
X=(CH₂)_{p+q} or (CH₂)ₚ—O—(CH₂)_{q},
p, q are each, independently of one another, from 0 to 10 and
R, R′ are identical or different and are each, independently of one another, H, a linear or branched C₁-C₁₈ alkyl or alkoxy radical or a linear or branched C₁-C₁₈ alkylsulfonate or alkoxysulfonate radical and
R″=an oligonucleotide radical or a radical selected from the group consisting of metal-free or metal-containing porphyrins, functionalized ferrocenes and functionalized calixarenes.

9. A polythiophene as claimed in claim 8, characterized in that
m=1,
n=2 to 200, preferably from 2 to 20,
X=(CH₂)₅,
R,R′=H and
R″=an oligonucleotide radical or a radical selected from the group consisting of metal-free or metal-containing porphyrins, functionalized ferrocenes and functionalized calixarenes.
